(19) United States
(12) Patent Application Publication
(10) Pub. No.: US 2025/0195768 A1
(43) Pub. Date: Jun. 19, 2025

(54) SMART LIQUID DRUG ADMINISTRATION

(71) Applicant: Janssen Pharmaceuticals, Inc., Titusville, NJ (US)

(72) Inventors: Ryan Walsh, Downingtown, PA (US); Justin Mellinger, Conshohocken, PA (US); John Quinlan, Fort Washington, PA (US)

(21) Appl. No.: 18/841,723

(22) PCT Filed: Feb. 24, 2023
(86) PCT No.: PCT/IB2023/051741

§ 371 Date: Aug. 27, 2024

Related U.S. Application Data
(60) Provisional application No. 63/317,789, filed on Mar. 8, 2022.

(51) Int. Cl. A61M 5/172; A61M 5/142

(52) U.S. Cl. CPC A61M 5/1723; A61M 5/14212; A61M 2005/14208; A61M 2205/52

(57) ABSTRACT

In general, devices, systems, and methods of smart liquid drug administration are provided. In an exemplary embodiment, a pump is configured to receive a reservoir therein. The reservoir contains a drug and includes a data storage component configured to store data. The pump includes a reader configured to read data from the data storage component. The pump is configured to, with the reservoir received therein, cause the drug to be delivered from the pump to a patient.

### CROSS-REFERENCE TO RELATED APPLICATIONS

This application claims priority to U.S. provisional patent application No. 63/317,789, filed Mar. 8, 2022, the entire disclosure of which is hereby incorporated by reference as if set forth in its entirety herein.

### FIELD

The present disclosure generally relates to smart liquid drug administration.

### BACKGROUND

Pharmaceutical products (including large and small molecule pharmaceuticals, hereinafter “drugs”) are administered to patients in a variety of different ways for the treatment of specific medical indications. A pump is a type of drug administration device that can administer a liquid drug to the patient. Some pumps are wearable by a patient and can include a reservoir, such as a vial or a cartridge, that contains the liquid drug therein for delivery to the patient through a needle or cannula inserted into the patient.

Care must be taken when administering drugs to a patient to avoid adverse effects on the patient. For example, care must be taken not to administer more than a safe amount of the drug to the patient. Different patients can require different dosages of a drug. Additionally, a dose of drug to be delivered to a patient can change over the course of a patient's treatment. Pumps are traditionally configured to deliver a preset amount of a drug to a patient, so different patients and changing dose requirements for a patient may not be possible with such pumps. For another example, care must be taken to deliver a drug to the patient at a safe, comfortable rate. Different drugs can be more comfortably delivered at different rates. Pumps are traditionally configured to deliver a drug at a preset rate, so different delivery rates may not be possible with such pumps and patients may therefore feel discomfort. For yet another example, different reservoirs can contain different drug amounts when loaded into a pump, but pumps traditionally do not account for different drug volumes in reservoirs.

Further, when it is possible to utilize different drugs in a given drug administration device or system, there is an increased risk that an incorrect dosage regimen will be used for the drug that is actually being delivered to a patient.

Accordingly, there remains a need for improved drug administration.

### SUMMARY

In general, devices, systems, and methods of smart liquid drug administration are provided.

In one aspect, a drug administration system is provided that in one embodiment includes a first reservoir, a second reservoir, and a pump. The first reservoir contains a first volume of a first drug therein and includes a first data storage component storing data indicative of the first volume. The second reservoir has a same size as the first reservoir, contains a second volume of a second drug therein, and includes a second data storage component storing data indicative of the second volume. The second volume is different from the first volume. The pump is configured to receive each of the first and second reservoirs therein. The pump includes a reader configured to read the stored data from the first and second data storage components, a pumping assembly configured to drive the first drug from the pump for delivery to a patient and to drive the second drug from the pump for delivery to the patient, and control circuitry operably coupled to the reader and to the pumping assembly. The control circuitry is configured to receive first data from the reader indicative of the stored data read from the first data storage component, establish a first dosing regimen for delivery of the first drug using the received first data, cause the pumping assembly to drive the first drug from the pump based on the first dosing regimen, receive second data from the reader indicative of the stored data read from the second data storage component, establish a second dosing regimen for delivery of the second drug using the received second data, and cause the pumping assembly to drive the second drug from the pump based on the second dosing regimen.

The drug administration system can vary in any number of ways. For example, the control circuitry can be configured to establish the first dosing regimen prior to any delivery of the first drug from the pump to the patient, and the control circuitry can be configured to establish the second dosing regimen prior to any delivery of the second drug from the pump to the patient. For another example, the first reservoir can be configured to be removed from the pump before the second reservoir is received in the pump. For yet another example, the reader can include a Near Field Communication (NFC) reader, the first data storage component can include a first NFC tag, and the second data storage component can include a second NFC tag. For still another example, the reader can include a QR code scanner, the first data storage component can include a first QR code, and the second data storage component can include a second QR code. For yet another example, the first data storage component can include a first electrically erasable programmable read-only memory (EEPROM), and the second data storage component includes a second EEPROM. For another example, the first drug can be the same as the second drug. For still another example, the first drug can be different from the second drug. For yet another example, the first and second reservoirs can be configured to be pre-loaded into the pump. For still another example, the first and second reservoirs can be configured to be user-loaded into the pump. For another example, the first volume of the first drug can be delivered to the patient in the first dosing regimen, and the second volume of the second drug can be delivered to the patient in the second dosing regimen. For yet another example, the first volume of the first drug can be delivered to the patient in the first dosing regimen, and less than the second volume of the second drug can be delivered to the patient in the second dosing regimen.

In another embodiment, a drug administration system includes a pump and a reservoir. The pump includes a housing, a reader, and control circuitry. The reservoir is configured to contain a drug therein. The reservoir includes a data storage component configured to be preprogrammed, prior to the reservoir being received in the housing of the pump, with data indicating a dose amount of the drug to be delivered to a patient using the pump. The reader is configured to, with the reservoir received in the housing of the pump, read the data indicating the dose amount from the data storage component. The control circuitry is configured to receive data from the reader indicative of the read data indicating the dose amount. The control circuitry is configured to cause a dose of the drug, at the dose amount, to be delivered from the pump to the patient.

The drug administration system can have any number of variations. For example, the dose amount can be based on at least one of a pre-identified therapy and a pre-identified prescription of the drug for the patient. For another example, the dose amount can be based on a pre-identified weight of the patient. For still another example, the dose amount can be based on at least one of gender, ethnicity, and genetic makeup. For yet another example, the reader can include a Near Field Communication (NFC) reader, and the data storage component can include an NFC tag. For yet another example, the reader can include a QR code scanner, and the data storage component can include a QR code. For another example, the data storage component can include an EEPROM. For still another example, the control circuitry can be configured to cause one or more additional doses of the drug to be delivered from the pump to the patient, and each of the one or more additional doses of the drug can be at the dose amount. For another example, the reservoir can be configured to be non-removably received in the housing of the pump. For yet another example, the reservoir can be configured to be removably and replaceably received in the housing of the pump. For another example, the drug administration system can also include a second reservoir configured to contain a second drug therein, the second reservoir can include a second data storage component configured to be preprogrammed, prior to the second reservoir being received in the housing of the pump, with data indicating a second dose amount of the second drug to be delivered to the patient using the pump, the reader can be configured to, with the second reservoir received in the housing of the pump, read the data indicating the second dose amount from the second data storage component, the control circuitry can be configured to receive data from the reader indicative of the read data indicating the second dose amount, and the control circuitry can be configured to cause a dose of the second drug, at the dose second amount, to be delivered from the pump to the patient. For yet another example, the reservoir can be configured to be pre-loaded into the housing. For another example, the reservoir can be configured to be user-loaded into the housing.

In another embodiment, a drug administration system includes a reservoir and a pump. The reservoir is configured to contain a drug therein. The reservoir includes a data storage component configured to store data therein regarding the drug. The reservoir includes a first alignment mechanism. The pump is configured to receive the reservoir therein. The pump includes a reader, a second alignment mechanism, and control circuitry. The reader is configured to, with the reservoir received in the pump, read the stored data from the data storage component. The second alignment mechanism is configured to engage the first alignment mechanism. The engagement of the first and second alignment mechanisms is configured to ensure that the data storage component is positioned with an effective reading range of the reader. The control circuitry is configured to receive data from the reader indicative of the stored data read from the data storage component, establish a dosing regimen for delivery of the drug using the received data, and cause the drug to be delivered to a patient based on the dosing regimen.

The drug administration system can vary in any number of ways. For example, the reservoir cannot be fully received in the pump without the first and second alignment mechanisms being aligned. For another example, the reader can include a Near Field Communication (NFC) reader, and the data storage component can include an NFC tag. For yet another example, the reader can include a radio frequency identification (RFID) scanner, and the data storage component can include an RFID tag. For still another example, the data storage component can include an EEPROM. For another example, the position of the data storage component can maximize a number of electromagnetic field lines generated by the reader that pass through the data storage component. For still another example, one of the first and second alignment mechanisms can be a female member and the other of the first and second alignment mechanisms can be a male member configured to slide within the female member. For another example, the reservoir can be configured to be pre-loaded into the pump. For still another example, the reservoir can be configured to be user-loaded into the pump.

In another aspect, a drug administration method is provided that includes a reader of a pump reading stored data from first and second data storage components, control circuitry of the pump establishing a first dosing regimen and causing a pumping assembly of the pump to drive a first drug from a first reservoir in the pump based on the first dosing regimen, and the control circuitry establishing a second dosing regimen and causing the pumping assembly to drive a second drug from a second reservoir in the pump based on the second dosing regimen. The first reservoir contains a first volume of the first drug therein and includes the first data storage component storing data indicative of the first volume. The second reservoir has a same size as the first reservoir, contains a second volume of the second drug therein, and includes the second data storage component storing data indicative of the second volume. The second volume is different from the first volume. The control circuitry receives first data from the reader indicative of the stored data read from the first data storage component, establishes the first dosing regimen using the received first data, receives second data from the reader indicative of the stored data read from the second data storage component, and establishes the second dosing regimen using the received second data.

The drug administration method can vary in any number of ways. For example, the control circuitry can establish the first dosing regimen prior to any delivery of the first drug from the pump to the patient, and the control circuitry can establish the second dosing regimen prior to any delivery of the second drug from the pump to the patient. For another example, the drug administration method can also include removing the first reservoir from the pump before the second reservoir is received in the pump. For yet another example, the reader can include a Near Field Communication (NFC) reader, the first data storage component can include a first NFC tag, and the second data storage component can include a second NFC tag. For still another example, the reader can include a QR code scanner, the first data storage component can include a first QR code, and the second data storage component can include a second QR code. For yet another example, the first data storage component can include a first EEPROM, and the second data storage component includes a second EEPROM. For another example, the first drug can be the same as the second drug. For still another example, the first drug can be different from the second drug. For still another example, each of the first and second reservoirs can be pre-loaded into the pump. For yet another example, each of the first and second reservoirs can be user-loaded into the pump.

In another embodiment, a drug administration method includes a reader of a pump, with a reservoir received in a housing of the pump, reading data indicating a dose amount from a data storage component of the reservoir, and control circuitry of the pump causing a dose of the drug, at the dose amount, to be delivered from the pump to a patient. The reservoir contains the drug therein. The data storage component is preprogrammed, prior to the reservoir being received in the housing of the pump, with data indicating the dose amount of the drug to be delivered to the patient using the pump. The control circuitry receives data from the reader indicative of the read data indicating the dose amount.

The drug administration method can have any number of variations. For example, the dose amount can be based on at least one of a pre-identified therapy and a pre-identified prescription of the drug for the patient. For another example, the dose amount can be based on a pre-identified weight of the patient. For yet another example, the dose amount can be based on at least one of gender, ethnicity, and genetic makeup. For still another example, the reader can include a Near Field Communication (NFC) reader, and the data storage component can include an NFC tag. For yet another example, the reader can include a QR code scanner, and the data storage component can include a QR code. For another example, the data storage component includes an EEPROM. For still another example, the control circuitry can cause one or more additional doses of the drug to be delivered from the pump to the patient, and each of the one or more additional doses of the drug can be at the dose amount. For another example, the reservoir can be non-removably received in the housing of the pump. For yet another example, the reservoir can be removably and replaceably received in the housing of the pump. For still another example, the reservoir can be pre-loaded into the housing. For yet another example, the reservoir can be user-loaded into the housing.

For another example, the drug administration method can also include the reader of the pump, with a second reservoir received in the housing of the pump, reading data indicating a second dose amount from a second data storage component of the second reservoir, and the control circuitry of the pump causing a second dose of the second drug, at the second dose amount, to be delivered from the pump to the patient. The second reservoir can contain the second drug therein. The second data storage component can be preprogrammed, prior to the second reservoir being received in the housing of the pump, with data indicating the second dose amount of the second drug to be delivered to the patient using the pump. The control circuitry can receive data from the reader indicative of the read data indicating the second dose amount.

In another embodiment, a drug administration method includes engaging a first alignment mechanism of a reservoir and a second alignment mechanism of a pump and then causing the reservoir to be received in the pump. The reservoir contains a drug therein. The reservoir includes a data storage component storing data therein regarding the drug. The pump includes a reader and control circuitry. The reader, with the reservoir received in the pump, reads the stored data from the data storage component. The second alignment mechanism engages the first alignment mechanism. The engagement of the first and second alignment mechanisms ensures that the data storage component is positioned with an effective reading range of the reader. The control circuitry receives data from the reader indicative of the stored data read from the data storage component, establishes a dosing regimen for delivery of the drug using the received data, and causes the drug to be delivered to a patient based on the dosing regimen.

The drug administration method can vary in any number of ways. For example, the reservoir cannot be fully received in the pump without the first and second alignment mechanisms being aligned. For another example, the reader can include a Near Field Communication (NFC) reader, and the data storage component can include an NFC tag. For yet another example, the reader can include a radio frequency identification (RFID) reader, and the data storage component can include an RFID tag. For still another example, the data storage component can include an EEPROM. For another example, the position of the data storage component can maximize a number of electromagnetic field lines generated by the reader that pass through the data storage component. For still another example, one of the first and second alignment mechanisms can be a female member and the other of the first and second alignment mechanisms can be a male member that slides within the female member. For another example, the reservoir can be pre-loaded into the pump. For still another example, the reservoir can be user-loaded into the pump.

In still another example, a drug administration system, comprises a pump assembly, a reader, and control circuitry. The pump assembly is configured to drive a drug from a reservoir for delivery to a patient. The reader is configured to read, from a data storage component of the reservoir, configuration data for configuring an operating parameter of the drug administration system. The control circuitry is configured to set the operating parameter of the drug administration system to a first value based on the configuration data so that, when the pump assembly drives the drug from the reservoir, the drug administration system operates with the operating parameter set to the first value.

Yet still another example is a method of administering a drug with a drug administration system. The method comprises communicatively coupling a reader of the drug administration system with a data storage component of a reservoir of the drug administration system. A reader reads, from the data storage component of the reservoir, configuration data for configuring an operating parameter of the drug administration system. The operating parameter of the drug administration system is set to a first value based on the configuration data. The pump assembly of the drug administration system is operated to drive the drug from the reservoir such that the drug administration system operates with the operating parameter set to the first value.

### BRIEF DESCRIPTION OF DRAWINGS

The present invention is described by way of reference to the accompanying figures which are as follows:

FIG. 1 is a schematic view of one embodiment of a pump configured to deliver a liquid drug to a patient;

FIG. 2 is a perspective view of one embodiment of a reservoir including one embodiment of a data storage component;

FIG. 3 is a perspective view of another embodiment of a reservoir including another embodiment of a data storage component attached to a bottom of the reservoir;

FIG. 4 is a perspective view of the reservoir of FIG. 3 with the data storage component attached to a side of the reservoir;

FIG. 5 is a front view of one embodiment of a reader antenna;

FIG. 6 is a perspective view of one embodiment of a flexible printed circuit board assembly;

FIG. 7 is another perspective view of the flexible printed circuit board assembly of FIG. 6;

FIG. 8 is a schematic view of another embodiment of a pump configured to deliver a liquid drug to a patient and one embodiment of a reservoir configured to be received in the pump;

FIG. 9 is a schematic view of the pump of FIG. 8 with the reservoir received therein;

FIG. 10 is a schematic view of one embodiment of relative positioning of a Near Field Communication (NFC) tag antenna and an NFC tag reader;

FIG. 11 is a schematic view of the relative positioning of FIG. 10 and embodiments of alternate relative positioning;

FIG. 12 is a schematic cross-sectional view of another embodiment of a reservoir received in a pump with alignment mechanisms of the reservoir and the pump engaged with one another and with a data storage component of the reservoir positioned relative to a reader of the pump;

FIG. 13 is a schematic cross-sectional view of another embodiment of a reservoir received in a pump that includes a multiplexed reader;

FIG. 14 is a schematic view of another embodiment of a pump configured to deliver a liquid drug to a patient;

FIG. 15 is a perspective view of yet another embodiment of a pump configured to deliver a liquid drug to a patient; and

FIG. 16 is a schematic view of yet still another embodiment of a pump configured to deliver a liquid drug to a patient.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

In general, devices, systems, and methods of smart liquid drug administration are provided. In an exemplary embodiment, a pump is configured to receive a reservoir therein. The reservoir, e.g., a vial, cartridge, etc., contains a drug and includes a data storage component configured to store data. The pump includes a reader configured to read data from the data storage component. The pump is configured to, with the reservoir received therein, cause the drug to be delivered from the pump to a patient.

The data read from the data storage component can be used by the pump in controlling drug delivery from the pump. The pump can therefore be configured to deliver drugs differently in different circumstances, depending on the stored data associated with the particular reservoir received by the pump. Multiple pumps may thus be similarly manufactured with each having a same stock-keeping unit (SKU) even though different ones of the pumps will eventually be used to deliver different drugs and/or to deliver the same or different drugs differently. Traditionally, pumps are manufactured with a different SKU for different pump uses, e.g., a first SKU for pumps to be used for a particular therapy, a second SKU for pumps to be used for a particular indication, etc. Manufacturing pumps with a single SKU instead of manufacturing pumps with multiple different SKUs may reduce costs and/or ease inventory management and shipping/tracking. Different reservoirs usable with such pumps can have different SKUs to facilitate programming of the pump in which the reservoir is received. Manufacturing reservoirs with different SKUs may be less complicated and less expensive than manufacturing pumps with different SKUs for various reasons, such as pumps being more complex technology to manufacture than reservoirs, reservoirs being produced in greater numbers than pumps, etc.

The data read from the data storage component being used in controlling drug delivery from the pump may simplify complex therapies such as high volume therapies and multiple therapies. The pump does not need to be manually programmed to ensure that the therapy is properly provided, which may help reduce error(s) in providing the therapy.

In some embodiments, the pump can be configured to receive only one reservoir therein. The reservoir may not be replaceable, e.g., not removable from the pump, in which case the pump cannot deliver any more drug(s) once the drug in the reservoir has been completely delivered (or the patient's treatment using the drug has ended without all of the drug in the reservoir having been delivered to the patient). Alternatively, once the drug contained in the reservoir has all been delivered from the pump to a patient (or the patient's treatment using the drug has ended without all of the drug in the reservoir having been delivered to the patient), the reservoir is configured to be replaced with another reservoir for the pump to be able to deliver any more drug to the patient.

In some embodiments, the pump can be configured to receive at least two reservoirs therein such that the pump has, at the same time, at least two reservoirs contained therein. Each of the at least two reservoirs can include a data storage component each readable by the reader of the pump, which may be one or more readers. In this way, the data read from a particular one of the data storage components can be used by the pump in controlling delivery of the drug from that particular reservoir. The pump may therefore control drug delivery differently for different ones of the reservoirs and the drugs contained therein. Similar to that discussed above, each of the reservoirs may be replaceable or non-replaceable.

In some embodiments, the data storage component for each reservoir receivable in the pump can store data thereon related only to its associated reservoir. In some embodiments, the data storage component for a first reservoir received in the pump can store data thereon related to at least one additional reservoir to be received in the pump. Each of the reservoirs can be sequentially received in the pump, e.g., in scenarios in which the pump is configured to receive only a single reservoir therein, or can be simultaneously contained in the pump, e.g., in scenarios in which the pump is configured to simultaneously contain therein two or more reservoirs. For example, in a dual therapy scenario in which a first drug is delivered to the patient followed by a second drug being delivered to the patient, the data can indicate that a second reservoir to be subsequently received in the pump should contain a particular drug or a particular type of drug therein. The pump can use this data after receiving the second reservoir therein to verify that the second reservoir contains the particular drug or the particular type of drug therein, e.g., by comparing the data read from the first reservoir's data storage component with data read from the second reservoir's data storage component. If the second reservoir is determined by the pump to not contain the expected drug or expected drug type, the pump can be configured to enter an error state in which the pump cannot deliver the drug contained in the reservoir since the drug is not the expected drug or expected drug type.

The drug to be delivered using a pump as described herein can be any of a variety of drugs. Examples of drugs that can be delivered using a pump as described herein include antibodies (such as monoclonal antibodies), hormones, antitoxins, substances for the control of pain, substances for the control of thrombosis, substances for the control of infection, peptides, proteins, human insulin or a human insulin analogue or derivative, polysaccharide, DNA, RNA, enzymes, oligonucleotides, antiallergics, antihistamines, anti-inflammatoirecorticosteroids, disease modifying anti-rheumatic drugs, erythropoietin, and vaccines.

The smart drug administration described herein can be used with a variety of drug delivery pumps configured to deliver a drug to a patient. The pumps can be “on body” pumps configured to be removably attached to a patient, or the pumps can be “off body” pumps configured to deliver a drug to a patient through an infusion set extending between the pump and the patient. Various embodiments of drug delivery pumps include the pumps described in, for example, Intl. Pat. Pub. WO 2021/124002 entitled “Liquid Drug Pumps Including User Feedback Indicating Pump Orientation” published Jun. 24, 2021, Intl. Pat. Pub. WO 2021/123995 entitled “Liquid Drug Pumps With A Flexible Drug Reservoir” published Jun. 24, 2021, Intl. Pat. Pub. WO 2021/059202 entitled “Drug Administration Device And System For Establishing a Dosing Regimen And Compatibility Of Components” published Apr. 1, 2021, Intl. Pat. Pub. WO 2021/059203 entitled “Drug Administration Devices That Communicate With External Systems And/Or Other Devices” published Apr. 1, 2021, Intl. Pat. Pub. WO 2021/059214 entitled “Drug Administration System Configured To Determine A Drug Dosing Scheme” published Apr. 1, 2021, Intl. Pat. Pub. WO 2018/096534 entitled “Apparatus For Delivering A Therapeutic Substance” published May 31, 2018, in U.S. Pat. Pub. No. 2019/0134295 entitled “Local Disinfection For Prefilled Drug Delivery System” published May 9, 2019, in U.S. Pat. No. 7,976,505 entitled “Disposable Infusion Device Negative Pressure Filling Apparatus And Method” issued Jul. 12, 2011, and in U.S. Pat. No. 7,815,609 entitled “Disposable Infusion Device Positive Pressure Filling Apparatus And Method” issued Oct. 19, 2010, which are hereby incorporated by reference in their entireties. Other examples of drug delivery pumps include the SmartDose® Drug Delivery Platform available from West Pharmaceutical Services, Inc. of Exton, PA, the OMNIPOD® available from Insulet Corp. of Acton, MA, the YpsoDose® patch injector available from Ypsomed AG of Burgdorf, Switzerland, the BD Libertas™ wearable injector available from Becton, Dickinson and Co. of Franklin Lakes, NJ, the Sorrel Medical pump available from Sorrel Medical of Netanya, Israel, the SteadyMed PatchPump® available from SteadyMed Ltd. of Rehovot, Israel, the Sensile Medical infusion pump available from Sensile Medical AG of Olten, Switzerland, the SonceBoz wearable injectors available from SonceBoz SA of Sonceboz-Sombeval, Switzerland, enFuse® available from Enable Injections of Cincinnati, OH, the on-body injector for Neulasta® available from Amgen, Inc. of Thousand Oaks, CA, the Pushtronex® System available from Amgen, Inc. of Thousand Oaks, CA, and the Imperium® pump available from Unilife Corp. of King of Prussia, PA.

Although the smart drug administration described herein is described with respect to drug delivery pumps, the smart drug administration can be used similarly with other drug administration devices configured to deliver a liquid drug to a patient, such as autoinjectors and jet injectors. Various embodiments of drug administration devices configured to deliver a liquid drug are described, for example, in previously mentioned WO 2021/059202 entitled “Drug Administration Device And System For Establishing a Dosing Regimen And Compatibility Of Components” published Apr. 1, 2021, Intl. Pat. Pub. WO 2021/059203 entitled “Drug Administration Devices That Communicate With External Systems And/Or Other Devices” published Apr. 1, 2021, and Intl. Pat. Pub. WO 2021/059214 entitled “Drug Administration System Configured To Determine A Drug Dosing Scheme” published Apr. 1, 2021.

FIG. 1 illustrates one embodiment of a pump 20, e.g., a patch pump or other pump, configured to deliver a liquid drug (also referred to herein as a “therapeutic substance”) 22 to the patient. The pump 20 includes a therapeutic substance reservoir 24 containing the drug 22 therein. The pump 20 can be configured to be removably attached to the patient in any of a variety of ways, as will be appreciated by a person skilled in the art, such as by including a backing or label configured to be removed from a body of the pump 20 to expose adhesive attachable to the patient. Alternatively, the pump 20 can be configured as an “off body” pump, as discussed further below.

The reservoir 24 in an exemplary embodiment is a vial, but the reservoir 24 can have other forms, such as a cartridge, a syringe, or a bag. The reservoir 24 is configured to be received in the pump 20, e.g., in a housing of the pump 20, to allow the drug 22 held in the reservoir 24 to be administered to the patient via the pump 20. The reservoir 24 when fully inserted into the pump 20 can be fully contained within the pump 20, e.g., fully contained within the pump's housing, or can be partially contained within the pump 20, e.g., a first portion of the reservoir 24 is located inside the housing and a second portion of the reservoir 24 is located outside of the housing. In some embodiments, the reservoir 24 is pre-loaded into the pump 20 by being installed in the pump 20 at a time of manufacture. A user can therefore receive the pump 20 with the reservoir 24 already received therein, which may ease the user's use of the pump 20 and/or help ensure that the correct drug 22 is delivered to a patient by the pump 20. The reservoir 24 in such embodiments can be non-removably received in the pump 20 such that the pump 20 can be a one-time use pump 20 configured to deliver only the drug 22 in the reservoir 24 (through one or more separate doses of the drug 22). In other embodiments, the reservoir 24 is user-loaded into the pump 20 by being installed in the pump 20 by the user. The reservoir 24 in such embodiments can be non-removably received in the pump 20 such that the pump 20 can be a single-use pump 20, or can be removably and replaceably received in the pump 20 such that the pump 20 can be a reusable, multi-use pump 20 configured to be used with each of a plurality of reservoirs received in the pump 20.

In an exemplary embodiment, the reservoir 24 is prefilled by a medical vendor or device manufacturer prior to the reservoir 24 being loaded into pump 20 and thus prior to use of the pump 20.

The pump 20 in this illustrated embodiment is configured to receive a single reservoir 24 at a time. In other embodiments, a pump can be configured to receive a plurality of reservoirs at a time.

The reservoir 24 includes a data storage component 48 that is attached to the reservoir 24, such as by being adhered to the reservoir 24 or embedded in the reservoir 24, or that is otherwise part of the reservoir 24, such as by being printed thereon. The data storage component 48 is configured to store data regarding the reservoir 24 and/or regarding the drug 22 contained in the reservoir 24. The data storage component 48 can have a variety of configurations. For example, the data storage component 48 can include an integrated circuit configured to communicate the reservoir data from the reservoir. One example of an integrated circuit is a near field communication (NFC) tag, also referred to as a proximity-integrated circuit card (PICC). An ISO14443-A passive NFC tag, an ISO15693 passive NFC tag, an ISO18000-3 passive NFC tag, an ISO14443-A/B passive NFC tag, a passive FeliCa NFC tag, or other type of NFC tag (passive or active) can be used. For another example, the data storage component 48 can include a radio frequency identification (RFID) tag. For yet another example, the data storage component can be in the form of a barcode. One example of a barcode is a QR code. Another example of a barcode is a Universal Product Code (UPC) code.

The reservoir 24 includes a single data storage component 48 in this illustrated embodiment but can include a plurality of data storage components. If a plurality of data storage components are used, each can be different from one another, which may help provide redundancy and/or allow for data retrieval even if a certain type of data communication is currently unavailable, e.g., if an RFID tag is absent or damaged so as to be unreadable a QR code may still be read.

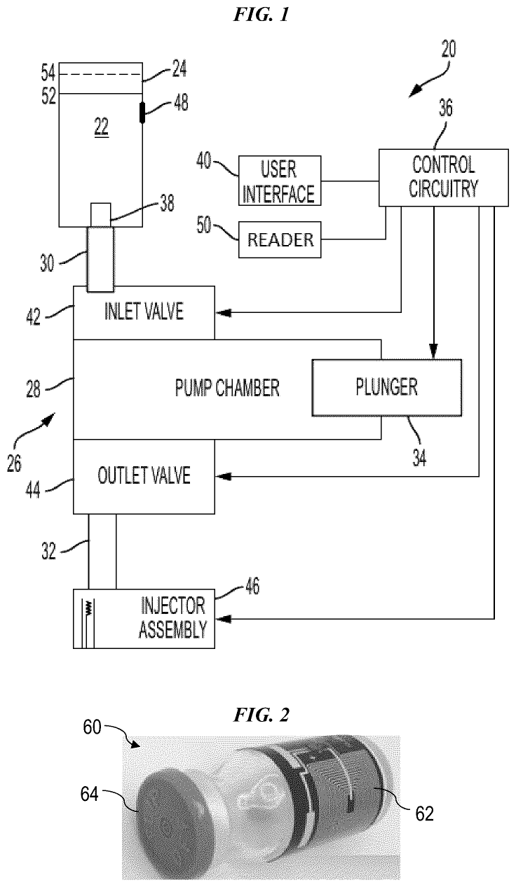

FIG. 2 illustrates one embodiment of a reservoir that can be used as the reservoir 24 having the data storage component 48 attached thereto. The reservoir is in the form of a vial 60 having a data storage component in the form of an NFC tag 62 on a flexible substrate adhered to an exterior of the vial 60. The NFC tag 62 is located on a side of the vial 60 in this illustrated embodiment but can be located elsewhere, such as on a top of the vial 60 or on a bottom of the vial 60. It will be understood that the data storage component could be a data storage component other than an NFC tag. The top of the vial 60 is obscured in FIG. 2 by a removable protective cap 64 configured to be removed from the vial 60 before the vial 60 is inserted into the pump 20 (or other pump or drug administration device). A location of the NFC tag 62 (or other data storage component on the vial 60 or other reservoir) can be selected to correspond to a location of the reader 50 in the pump 20 to facilitate the reader 50 being within effective reading range of the NFC tag 62. The NFC tag 62 has a square shape in this illustrated embodiment but can have another shape.

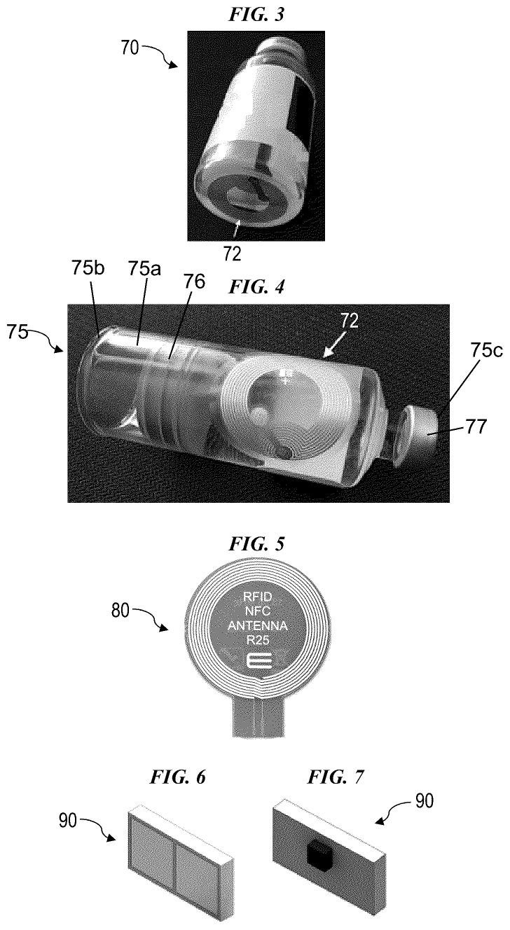

FIG. 3 illustrates another embodiment of a reservoir that can be used as the reservoir 24 having the data storage component 48 attached thereto. The reservoir is in the form of a vial 70 having a data storage component in the form of an NFC tag 72 on a flexible substrate adhered to an exterior of the vial 70. The NFC tag 72 in this illustrated embodiment is located on a bottom of the vial 70 and has a circular shape. It will be understood that the data storage component could be a data storage component other than an NFC tag.

FIG. 4 illustrates another embodiment of a reservoir in which the reservoir is in the form of a container 75. The container 75 is shown as a cartridge but could alternatively be a syringe. The container 75 has a container body 75a for containing the drug therein. The data storage component is in the form of an NFC tag 72 located on a side of the container body 75a. It will be understood that the data storage component could be a data storage component other than an NFC tag. The container body 75a defines a cavity configured to hold a liquid drug therein. The container body 75a has a first end 75b and a second end 75c. The container body 75a can have a central axis that extends along an axial direction. The first end 75b can define an opening therein that is open to the cavity. The drug container 75 can comprise a seal 76 disposed in the cavity that forms a seal with an interior surface of the container body 75a. The seal 76 can be received through the opening in the first end 75b into the cavity. The seal 76 is configured to translate towards the second end 75c to drive the liquid drug from the cavity.

In some examples, such as where the drug container 75 is a cartridge, the drug container 75 can comprise a cap 77 on the second end 75c. The cap 77 can be formed from any suitable material, such as a metal. The cap 77 can be crimped onto a head of the container body 75a at the second end 75c. The drug container 75 can comprise a septum (not shown) that is configured to seal the second end 75c. The septum can be supported by the cap 77 and can be configured to be pierced by a piercing needle to open a fluid path into the drug container 75.

Referring again to FIG. 1, the pump 20 also includes a reader 50 configured to read the data storage component 48. In general, a type of the reader 50 depends on the form of the data storage component 48 so that the reader 50 can acquire data from the data storage component 48 since acquiring data from the data storage component 48 requires the use of an appropriate communications interface for receiving the data from the data storage component 48. For example, when the data storage component 48 includes an RFID tag, the reader 50 includes an RFID scanner. For another example, when the data storage component 48 includes a barcode, the reader 50 includes a barcode scanner. For yet another example, when the data storage component 48 includes an integrated circuit, the integrated circuit can be powered and capable of transmitting the data to a receiving communications interface, such as an NFC reader configured to read an RFID tag. The reader is configured to communicatively couple with the data storage component when the reader and data storage component are brought within proximity to one another so that the reader and data storage component communicate. In some examples, the reader is configured to communicatively couple with the data storage component when the reservoir is received in a housing of the pump, but not before then. In some such examples, the reservoir must be received in the housing of the pump in a specific orientation in order for the reader to communicatively couple with the data storage component.

In some examples, the reader 50 can transmit data as well as receive data, and optionally cause that data to be written to the data storage component 48. In some such examples, the data storage component 48 can be updated at regular intervals (e.g., at delivery completion of each mL) so that it contains a reasonably-accurate record of the delivery progress at any given time. If the pump 20 should fail during delivery, resulting in a partially-delivered dose, the reservoir containing a record of the partial dose can be transferred to a secondary pump, where the remaining dose could be delivered. In some such examples, the data storage component 48 can also be updated with any information relevant to the delivery and pump state during delivery.

For example, the information can include the date and time of delivery, the model and serial number of the pump, the ambient pump and reservoir temperatures, pump user input settings, pump wireless communications events, pump warning or alarm events, user-initiated pauses and durations, user interface events, and/or relevant pump parameters settings and measurements during delivery (force, pressure, battery voltage/current, etc.). As such, the data storage component 48 could serve as a record of the delivery (e.g., a delivery “black-box” recording). The data storage component 48 can be designed to easily peel off the reservoir, so that it can be transferred to a monitoring party or HCP for subsequent reading, recording and analysis. Upon completion of delivery, the data storage component 48 can be updated with a “delivery-completed” status, thus preventing the reservoir from being refilled and reused in pumps, including those with capabilities described by the disclosure.

In an exemplary embodiment the reader 50 is a single reader, which may help reduce cost of the pump 20, help conserve space within the pump 20 for other components, and/or help reduce an overall size of the pump 20. The reader 50 can instead include a multiplexed reader, which may allow for a one of the readers with the best communication with the data storage component 48 to be the one used, and/or may help provide redundancy and allow for read data to be confirmed with one another for accuracy.

The reader 50 is configured to read data from the data storage component 48 with the data storage component 48 being within an effective reading range of the reader 50 and the reader 50 being “on.” The reader 50 is “on” when the reader 50 is sufficiently powered to read data. In an exemplary embodiment, the pump 20 being powered on is configured to power on the reader 50. The reader 50 can thus be configured to read data from the data storage component 48 upon powering on of the pump 20 if the reservoir 24 has already been inserted into the pump 20 such that the data storage component 48 is within an effective reading range of the reader 50. The reservoir 24 is typically inserted into the pump 20 with the pump 20 powered off or in a low power or power conservation mode. Powering on the pump 20 can thus trigger the reader 50 to automatically read data from the data storage component 48. The reader 50 can therefore be configured to read data from the data storage component 48 prior to a first delivery of the drug 22 from the pump 20 because the pump 20 cannot deliver the drug 22 to a patient with the pump 20 powered off or in a low power or power conservation mode.

The pump 20 can be powered on by a user of the pump 20, thereby allowing data to be read from the data storage component 48 after the user has received the pump. In such embodiments, the reservoir 24 may have been user-loaded or pre-loaded. Alternatively, in embodiments in which the reservoir 24 is pre-loaded into the pump 20 during manufacturing, the pump 20 can be powered on during manufacturing, thereby allowing data to be read from the data storage component 48. Data may thus be read from the pump 20 before a user receives the pump 20. After data has been read from the data storage component 48, the pump 20 can be powered off or put in a low power or power conservation mode to await being later turned on by a user.

In one embodiment, the reader 50 can include a printed circuit board (PCB) having mounted thereon an NFC transceiver, such as a DLP7970A NFC transceiver available from DLP Design Inc. of Mckinney, TX or other type of NFC transceiver, and a microcontroller, such as a MSP430G2553 microcontroller available from Texas Instruments, Inc. of Dallas, TX or other type of microcontroller. The DLP7970A transceiver is configured to read passive NFC tags. In such an embodiment, the reader 50 is not configured to directly contact the data storage component 48.

FIG. 5 illustrates one embodiment of a reader antenna 80 that can be used as the reader 50. The reader antenna 80 in this illustrated embodiment is an RFID/NFC R25 reader antenna configured for use with RFID tags and with NFC tags. The reader antenna 80 in this illustrated embodiment is on a thin polyester or polyimide flex film substrate, which may facilitate reading of an RFID tag or an NFC tag that is on a curved surface of a reservoir, such as on a cylindrical side of a reservoir as shown for example with the reservoirs 60, 70 of FIGS. 2 and 4, because the reader antenna 80 can be attached to a curved surface within the pump 20 corresponding to the data storage component's curvature on a side of the reservoir 24 to facilitate the reader's positioning with respect to the data storage component 48. In this illustrated embodiment, the reader 50 is not configured to directly contact the data storage component 48.

FIGS. 6 and 7 illustrate one embodiment of a flexible printed circuit board assembly (PCBA) 90 that can be used as the reader 50. The PCBA 90 is configured to read a passive NFC tag. The PCBA 90 includes a one-wire electrically erasable programmable read-only memory (EEPROM) with exposed pads. A spring contacts the main PCB of the PCBA 90 to read from the one-wire EEPROM. In this illustrated embodiment, the reader 50 is configured to directly contact the data storage component 48.

Referring again to FIG. 1, the pump 20 includes control circuitry 36 configured to control administration of the drug 22 from the pump 20 based at least in part on data received from the reader 50 so as to be based at least in part on the data stored on the data storage component 48 of the reservoir 24 and read by the reader 50. In some embodiments, the reader 50 is configured to automatically read data from the data storage component 48 and transmit to the control circuitry 36 data indicative of the data read from the data storage component 48. In other embodiments, the control circuitry 36 is configured to transmit an instruction to the reader 50, e.g., upon powering on of the pump 20, to read data from the data storage component 48 and transmit to the control circuitry 36 data indicative of the data read from the data storage component 48.

The control circuitry 36 is configured to control administration of the drug 22 from the pump 20 according to a dosing regimen. The dosing regimen refers to the specific manner in which the drug is delivered, including (without limitation) formulation, route of administration, dose interval (frequency of dosing), dose amount or volume, delivery rate (flow rate), delivery duration, pauses in delivery, pauses between delivery phases in a multi-drug delivery sequence, and sequencing order of a multi-drug delivery sequence. The dosing regimen can be stored as an algorithm in a memory of the control circuitry 36 that a processor of the control circuitry 36 is configured to execute. The algorithm is stored in the form of one or more sets of pluralities of data points defining and/or representing instructions, notifications, signals, etc. to control administration of a drug from the pump 20.

The algorithm includes one or more variable parameters that can be changed by the control circuitry 36 such that the dosing regimen can be changed. The variable parameters are among the algorithm's data points and are thus each able to be changed by changing one or more of the stored pluralities of data points of the algorithm. The control circuitry 36, e.g., the processor thereof, is configured to establish at least one of the one or more variable parameters based on the data received from the reader 50 so as to be based at least in part on the data stored on the data storage component 48 of the reservoir 24. The data received from the reader 50 by the control circuitry 36 identifies the variable parameter(s) to establish and each variable parameter's value to allow the control circuitry 36 to update the algorithm to include the variable parameter values indicated by the data. After the dosing parameters have been established, subsequent execution of the algorithm by the control circuitry 36, e.g., the processor thereof, administers a dose of the drug 22 according to the algorithm, which reflects the current dosing regimen. The drug 22 can therefore be administered to the patient on a customized basis, as different data storage components will store different variable parameter values based on the particular drug 22, based on a particular therapy in which the drug 22 is being used to treat a particular disorder, and/or the particular patient to whom the drug 22 is intended to be delivered using the pump 20. Patient outcomes may thereby be improved by allowing the pump 20 to deliver the drug 22 according to requirements of a particular therapy, a particular drug, and/or a particular patient and/or by facilitating personalized medicine. The pump 20 may thus be able to be manufactured in a generic fashion and be later programmed to take into account requirements of a particular therapy, a particular drug, and/or a particular patient. The supply chain may thus be simplified and made more cost effective.

The pump 20 also includes a conduit 38 through which the drug 22 is configured to pass from the reservoir 24 and into an inlet fluid path 30 operatively connected to an injector assembly 46 of the pump 20 that is configured to deliver the drug 22 into a patient. The conduit 38 is thus a tube in which the drug 22 can flow. Instead of being an “on body” pump configured to be removably attached to the patient, the pump 20 can be an “off body” body pump configured to deliver a drug to a patient through an infusion set extending between the pump and the patient. The “off body” pump is generally configured and used similar to the pump 20 except that the injector assembly 46 can be configured differently or replaced with another type of assembly for use with the infusion set.

The pump 20 also includes a user interface 40 configured to provide information to a user of the pump 20, e.g., the patient associated with the pump 20 (e.g., wearing the pump 20 by having the pump removably attached thereto using adhesive or other attachment mechanism, or being operatively connected to the pump 20 via an infusion set), the patient's care giver assisting the patient in using the pump 20, a health care professional assisting the patient in using the pump 20, etc. The user interface 40 can have a variety of configurations, and the pump 20 can include a single type of user interface or can include more than one type of user interface. For example, the user interface 40 can include one or more lights, e.g., a light emitting diode (LED) or other type of light, configured to illuminate to provide various information. Examples of the information indicated by the user interface 40 include power (on/off) status, error state (e.g., indication that an error in the pump 20 has been detected such as low power supply, improper needle advancement into the patient, incompatible type of reservoir 24 loaded into the pump 20, etc.), drug delivery status (e.g., indication that drug delivery is currently occurring), drug delivery progress information, an orientation of the pump 20 relative to gravity, an indication of a dose of the drug 22 to be provided in each delivery of the drug 22 to the patient, and other types of information. For another example, the user interface 40 can include a display configured to show information thereon, such as by using text and/or graphics. The display can include a display screen having any of a variety of configurations, such as a cathode ray tube (CRT), a liquid crystal display (LCD), a touchscreen, etc. For yet another example, the user interface 40 can include a vibration mechanism configured to vibrate with the vibration being configured to be felt by the patient wearing the pump 20. For still another example, the user interface 40 can include a speaker configured to provide an audio signal. For another example, the user interface 40 can include a mechanical level configured to indicate the pump's orientation.

The control circuitry 36 is operatively coupled to the user interface 40 and is configured to cause the user interface 40 to provide information to the user. The control circuitry 36 is also operatively coupled to the reader 50 and is configured to receive data from the reader 50 indicative of the data read from the data storage component 48, e.g., to receive a signal from the reader 50 indicative of the read data. The control circuitry 36 is configured to control the pump 20 using the data received from the reader 50, as discussed further herein.

An electromechanical pumping assembly 26, e.g., a motor thereof, is operatively coupled to the reservoir 24 and is configured to cause delivery of the therapeutic substance 22 to the patient via the injector assembly 46, e.g., through a needle or cannula of the injector assembly 46 that has been inserted into the patient. The electromechanical pumping assembly 26 is shaped to define a rigid pump chamber 28 that includes a therapeutic substance inlet 30 through which the therapeutic substance 22 is received from the conduit 30, and hence from the reservoir 24, into the pump chamber 28. The rigid pump chamber 28 also includes a fluid path outlet 32 through which the therapeutic substance 22 is delivered from the pump chamber 28 to the patient via the injector assembly 46. Although the pumping assembly 26 is electromechanical in this illustrated embodiment, the pumping assembly of the pump 20 (and for other embodiments of pumps described herein) can instead be any suitable alternative pumping assembly, such as a mechanical pumping assembly (e.g., with spring), pneumatic pumping assembly, or hydraulic pumping assembly. The mechanical pumping assembly need not include any electronic components or controls. For example, the mechanical pumping assembly can include a balloon diaphragm configured to be activated to cause delivery of a drug through mechanical action.

The pump 20 also includes a plunger 34 slidably disposed within the pump chamber 28 and sealably contacting an inside of the pump chamber 28. The plunger 34 is configured to be in direct contact with the drug 22 in the pumping chamber 28.

The electromechanical pumping assembly 26 in this illustrated embodiment is configured to be driven to operate in two pumping phases by the control circuitry 36. In other embodiments, the control circuitry 36 can be configured to drive the electromechanical pumping assembly 26 in another way, such as by operating in a single phase, by operating in more than two phases, or by operating with first and second pumping phases configured differently than the first and second pumping phases discussed below.

In a first pumping phase, the control circuitry 36 is configured to drive the plunger 34 (e.g., slidably move the plunger 34 in the pump chamber 28) to draw the drug 22 from the reservoir 24 into the conduit 38, then into the inlet fluid path 30, and then through an inlet valve 42 and into the pump chamber 28. The inlet valve 42 is configured to be opened and closed such that when the inlet valve 42 is open there is fluid communication between the reservoir 24 and the pump chamber 28, and when the inlet valve 42 is closed there is no fluid communication between the reservoir 24 and the pump chamber 28. During the first pumping phase, the control circuitry 36 is configured to cause the inlet valve 42 to open, cause an outlet valve 44 to close, and drive the plunger 34 to draw the therapeutic substance 22 from the reservoir 24 into the pump chamber 28, e.g., the control circuitry 36 is configured to set the inlet valve 42 and the outlet valve 44 such that the therapeutic substance 22 can flow only between the reservoir 24 and the pump chamber 28. Thus, as the plunger 34 is drawn back, the therapeutic substance 22 is drawn into pump chamber 28. The control circuitry 36 causing the inlet valve 42 to open and the outlet valve 44 to close can be active control or can be passive control in which the valves 42, 44 are mechanical valves that automatically open/close due to the driving of the plunger 34.

In a second pumping phase, the control circuitry 36 is configured to drive the plunger 34 to deliver the drug 22 from the pump chamber 28 through the outlet valve 44 to the outlet fluid path 32 and then to the injector assembly 46 for delivery into the patient. The outlet valve 44 is configured to be opened and closed such that when the outlet valve 44 is open there is fluid communication between the pump chamber 28 and the patient, and when the outlet valve 44 is closed there is no fluid communication between the pump chamber 28 and the patient. During the second pumping phase, the control circuitry 36 is configured to cause the inlet valve 42 to close, cause the outlet valve 44 to open, and drive the plunger 34 to deliver the therapeutic substance 22 from the pump chamber 28 in a plurality of discrete motions of the plunger 34. For example, the control circuitry 36 can be configured to set the inlet valve 42 and the outlet valve 44 such that the therapeutic substance 22 can flow only between the pump chamber 28 and the patient, and the plunger 34 is incrementally pushed back into the pump chamber 28 in a plurality of discrete motions thereby delivering the therapeutic substance 22 to the patient in a plurality of discrete dosages. Similar to that discussed above, the control circuitry 36 causing the inlet valve 42 to close and the outlet valve 44 to open can be active control or can be passive control in which the valves 42, 44 are mechanical valves that automatically open/close due to the driving of the plunger 34.

In some embodiments, the control circuitry 36 is configured to drive the plunger 34 to draw the therapeutic substance 22 into the pump chamber 28 in a single motion of the plunger 34, e.g., the plunger 34 is pulled back in a single motion to draw a volume of the therapeutic substance 22 into the pump chamber 28 during the first pumping phase. Alternatively, the control circuitry 36 can be configured to drive the plunger 34 to draw the therapeutic substance 22 into the pump chamber 28 in one or more discrete expansion motions of the plunger 34, e.g., the plunger 34 can be pulled halfway out of the pump chamber 28 in one motion and then the rest of the way out of the pump chamber 28 in a second, separate motion. In this case, a duration of some or all expansion motions of the plunger 34 during the first pumping phase are typically longer than a duration of any one of the plurality of discrete motions of the plunger 34 during the second pumping phase.

In other embodiments, the control circuitry 36 is configured to drive the plunger 34 such that a duration of the first pumping phase and a duration of the second pumping phase are unequal. For example, a duration of the second pumping phase can be in a range of five to fifty times longer than the first pumping phase, e.g., at least ten times, thirty times, fifty times, etc. longer than a duration of the first pumping phase.

The pump 20 can also include a power supply (not shown) configured to provide power to components requiring power to operate, such as the control circuitry 36 and the reader 50. In an exemplary embodiment, the power supply is a single power supply configured to provide power to each component of the pump 20 requiring power to operate, which may help reduce cost of the pump 20, help conserve space within the pump 20 for other components, and/or help reduce an overall size of the pump 20. The power supply can, however, include a plurality of power supplies, which may help provide redundancy and/or help reduce cost of the pump 20 since some components may be manufactured with an on-board dedicated power supply. In an exemplary embodiment, the power supply is on-board the pump 20, which may facilitate use of the pump 20 at any time in any location. In other embodiments, the power supply can include a mechanism configured to connect the pump 20 to an external power supply.

An amount (e.g., volume) of the drug 22 that the pump 20 delivers to a patient per dose can vary per disorder and/or per patient. The dose amount can vary for a variety of reasons. For example, different drugs may be administered at different doses because, e.g., some drugs may risk more and/or stronger side effects at higher volumes than other drugs, some drugs are more concentrated than other drugs, etc. Thus, depending on the particular drug 22 held by the reservoir 24, the amount of the drug 22 the pump 20 should deliver to the patient per dose can vary. For another example, different patients can receive different amounts of a same drug per the particular patient's prescription. Thus, depending on the particular patient to whom the drug 22 is to be delivered from the reservoir 24, the dose amount may vary even though the pump 20 is the same. For still another example, depending on the disorder indicated for treating a particular disorder, the amount of the drug 22 the pump 20 should deliver to a patient per dose can vary. Thus, depending on the particular therapy for which the drug 22 is being used, the dose amount may vary even though a different dose amount may be used for other therapies involving delivery of the same drug 22. The disorder is an example of a pre-identified characteristic for a patient. For another example, a dose amount of the drug 22 can vary for the same patient even though the pump 20 remains the same because the patient's therapy can change over time. For still another example, depending on gender as a patient population factor, the dose amount may vary even though the pump 20 is the same. For another example, depending on ethnicity as a patient population factor, the dose amount may vary even though the pump 20 is the same. For still another example, depending on genetic makeup as a patient population factor, the dose amount may vary even though the pump 20 is the same. A patient having a particular genetic makeup, e.g., including a particular gene such as a tumor-suppressing gene, can influence how much drug a particular patient should receive. The reservoir 24 can include a sensor thereon configured to read a biomarker of the patient indicative of the genetic makeup. Data regarding the read biomarker can be included in data read from the date storage component 48. For yet another example, a dose amount can be based on a weight of the particular patient to whom the drug 22 is to be delivered from the reservoir 24. In such a weight-based dosing scheme, an amount of the drug 22 that is delivered to patient from the reservoir 24 per dose is calculated based on a weight of the particular patient. The patient's weight is an example of a pre-identified characteristic for a patient. Thus, because different patients have different weights, the dose amount can vary between patients even though the pump 20 is the same for the patients and even if the drug 22 is the same for the patients.

In at least some embodiments, the reservoir 24 is intended to be used with a particular patient such that the drug 22 contained in the reservoir 24 is intended to be delivered to a particular patient. The data storage component 48 is configured to store data indicating a dose amount of the drug 22 to be delivered to the particular patient from the pump 20 to which the reservoir 24 is operatively coupled. The data indicating the dose amount is pre-programmed into the data storage component 48 prior to the reservoir 24 being received in the pump 20. The data can be pre-programmed in a variety of ways, as appropriate for the particular type of data storage component 48. With the reservoir 24 operatively coupled to the pump 20, the pump's reader 50 is configured to read the pre-programmed dose amount data from the data storage component 48. As discussed herein, the reader 50 is configured to read the data storage component 48 to gather data therefrom and is configured to transmit data indicative of the read data to the control circuitry 36. As also discussed herein, the control circuitry 36, e.g., the processor thereof, is configured to adjust the algorithm stored in the control circuitry 36, e.g., the memory thereof, so as to control future delivery of the drug 22 from the pump 20 based at least in part on the data.

In at least some embodiments, the pre-programmed dose amount reflects a weight-based dosing scheme and is based on a weight of a particular patient who will use the pump 20. A weight-based dosing scheme may help ensure that the patient receives enough of the drug 22 per dose for the drug 22 to effectively provide the intended treatment without the patient receiving too much of the drug 22 so as to waste drug 22 and/or to increase risk and/or magnitude of side effects.

In at least some embodiments, the pre-programmed dose amount reflects a prescribed, non-weight-based dose amount for a particular patient who will use the pump 20. The pump 20 may therefore deliver an appropriate amount of the drug 22 to the patient to maximize effectiveness of a particular patient's treatment.

In at least some embodiments, the pre-programmed dose amount reflects a particular therapy to treat a particular disorder. The pump 20 may therefore deliver an appropriate amount of the drug 22 to a patient to maximize effectiveness of the therapy.

In at least some embodiments, the pre-programmed dose amount does not change with each dose of the drug 22 that the pump 20 delivers to a patient.

In at least some embodiments, the pre-programmed dose amount is different for different doses of the drug 22 that the pump 20 delivers to a patient. Over time, the patient may receive different amounts of the drug 22 from the pump 20 to maximize the patient's treatment using the drug 22. For example, some drugs are delivered in a split dosing arrangement in which an initial dose of the drug 22 at a first amount is delivered to the patient, at least one transition dose of the drug 22 at a second amount is delivered to the patient after the initial dose, and at least one maintenance dose of the drug 22 at a third amount is delivered to the patient after the transition dose(s). The first, second, and third amounts of the drug 22 are different from one another. The data stored on the data storage component 48, read by the reader 50, and communicated to the control circuitry 36 can reflect each of the first, second, and third amounts. The dosing regimen can thus be established such that the dose amount of the drug 22 changes over time as the initial dose (at the first dose amount), then the transition dose(s) (at the second dose amount), and then the maintenance dose(s) (at the third dose amount) are delivered to the patient via the pump 20.

In at least some embodiments, the data storage component 48 is configured to store data indicating a delivery rate of the drug 22 to be delivered from the pump 20 to which the reservoir 24 is operatively coupled. The delivery rate may vary, for example, from one therapy to another to account for drug viscosity. The data indicating the delivery rate is pre-programmed into the data storage component 48 prior to the reservoir 24 being received in the pump 20. The data can be pre-programmed in a variety of ways, as appropriate for the particular type of data storage component 48. With the reservoir 24 operatively coupled to the pump 20, the pump's reader 50 is configured to read the pre-programmed delivery rate data from the data storage component 48. As discussed herein, the reader 50 is configured to read the data storage component 48 to gather data therefrom and is configured to transmit data indicative of the read data to the control circuitry 36. As also discussed herein, the control circuitry 36, e.g., the processor thereof, is configured to adjust the algorithm stored in the control circuitry 36, e.g., the memory thereof, so as to control future delivery of the drug 22 from the pump 20 based at least in part on the data, e.g., so the drug 22 is delivered at the delivery rate read from the data storage component 48.

As mentioned above, the reservoir 24 can be removably and replaceably received in the pump 20 such that the pump 20 can be a reusable, multi-use pump 20 configured to be used with each of a plurality of reservoirs 24 received in the pump 20. Each of the reservoirs 24 receivable in the pump 20 includes its own data storage component 48 such that the pump 20 can control delivery of the drug 22 from each of the reservoirs 24 in a customized way with the pump 20 updating the dosing regimen based on a particular reservoir's stored data. A single pump 20 can thus be used to deliver drugs according to different dosing regimens. In some embodiments, each of the plurality of reservoirs 24 received in the pump 20 contains a same type of drug 22 therein such that the pump 20 can differently control delivery of the same type of drug 22 to one patient based on the data pre-programmed into each reservoir's data storage component 48. In other embodiments, one or more of the plurality of reservoirs 24 received in the pump 20 contain a different type of drug 22 therein than one or more of the other plurality of reservoirs 24 such that the pump 20 can deliver at least two different types of drugs 22 to one patient and the delivery of each of the different types of drugs 22 can be controlled based on the data pre-programmed into each reservoir's data storage component 48.

In embodiments in which the pump 20 is a reusable, multi-use pump 20 configured to be used with each of a plurality of reservoirs 24 received in the pump 20, each of the reservoirs 24 can be a same size as one another with each of the reservoirs 24 being configured to contain therein a same maximum amount of drug. Each of the reservoirs 24 having a same size may help ensure that the reservoirs 24 properly seat in the pump 20 such that the drug 22 contained therein can be in proper fluid communication with the injector assembly 46 of the pump 20. A same amount of drug 22 can be contained in each of the same-sized plurality of reservoirs 24. FIG. 1 shows a first fill level 52 of the drug 22 that can be used in each of the same-sized plurality of reservoirs 24. (The reservoir 24 is inverted in the view of FIG. 1 such that an upper end of the reservoir 24 is pointed downward.) It may be efficient and cost effective for multiple reservoirs 24 to be manufactured identically to one another each with a same amount of drug 22 loaded therein. However, not all patients will need to receive all of the drug 22 contained therein, e.g., due to different prescriptions, different patient weights, etc. Surplus drug 22 may thus be left in the reservoir 24 as not having been delivered to a patient. Thus, in some embodiments, a different amount of drug 22 can be contained in different ones of the same-sized plurality of reservoirs 24 such that at least one of the plurality of reservoirs 24 contains a different amount of drug 22 than at least one other of the plurality of reservoirs 24. A reservoir 24 containing an amount of drug 22 appropriate for a particular patient, particular therapy, etc. can thus be inserted into the pump 20. Drug may therefore not be left in the reservoir 24 as surplus to be discarded with the reservoir 24 while still allowing efficient, cost-effective manufacture of same-sized reservoirs 24 each configured to be usable with the pump 20. For example, one or more of the plurality of reservoirs 24 can have the first fill level 52, and one or more of the other plurality of reservoirs 24 can have a second, different fill level 54. One or more additional different fill levels may be used.

Fill levels can vary between sane-sized reservoirs 24 by any increment, such as by about 0.5 mL, about 1 mL, about 1.5 mL, about 2 mL, about 5 mL, etc. A person skilled in the art will appreciate that an amount may not be precisely at a value but nevertheless be considered to be at about that value due to any number of factors, such as manufacturing tolerances and sensitivity of measurement equipment. For example, the first and second fill levels 52, 54 can differ by about 0.5 mL, by about 1 mL, by about 1.5 mL, by about 2 mL, by about 5 mL, etc.

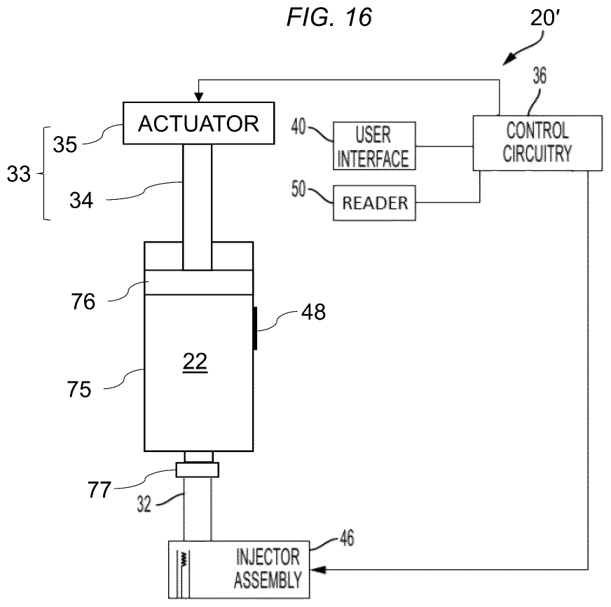

Referring briefly to FIG. 16, an alternative pump 20′, e.g., a patch pump or other pump, is shown that is configured to deliver a liquid drug 22 to the patient. The pump 20′ includes a therapeutic substance reservoir 75 containing the drug 22 therein. The pump 20′ can be configured to be removably attached to the patient in any of a variety of ways, as will be appreciated by a person skilled in the art, such as by including a backing or label configured to be removed from a body of the pump 20′ to expose adhesive attachable to the patient. Alternatively, the pump 20′ can be configured as an “off body” pump.

The reservoir 24 in an exemplary embodiment is a cartridge such as that shown in FIG. 4 or a syringe. The pump 20′ comprises control circuitry 36, user interface 40, reader 50, and injector assembly 46, which can be configured as discussed above. The pump 20′ comprises a pumping assembly 33 having an actuator 35 and a plunger 34. The actuator 35, which is controlled by the control circuitry 36, is configured to cause the plunger 34 to translate the seal 76 of the container 75 within the container body 75 to drive the drug 22 out of the container 75. The drug is driven through a fluid path outlet 32 to the injector assembly 46, which delivers the drug to the patient. The actuator 35 of the pumping assembly can be any suitable actuator, such as (without limitation) a mechanical actuator, an electromechanical actuator, a pneumatic actuator, or a hydraulic actuator.

Although examples above describe the data storage component 48 as storing data such as volume, dose amount, and delivery rate (or flow rate), examples of the disclosure are not so limited. In various examples, the data stored by the data storage component of a reservoir can store any suitable data for configuring an operating parameter of the drug administration system, and the control circuitry 36 can be configured to set the operating parameter of the drug administration system to a first value based on the configuration data so that, when the pump assembly drives the drug from the reservoir, the drug administration system operates with the operating parameter set to the first value. In such manner, a data storage component can store information that is usable to configure the pump's hardware and software for preferred delivery of its associated drug. It will be understood that the control circuitry 36 can be configured to change the operating parameter of the drug administration system to one or more values based on the configuration data after setting the operating parameter to the first value, and the drug administration system can further operate with the operating parameter set to the one or more over values. Of course, the data storage component can store configuration data for configuring more than one operating parameter, such as a plurality of operating parameters, and the control circuitry 36 can be configured to set the operating parameters of the drug administration system based on the configuration data so that, when the pump assembly drives the drug from the reservoir, the drug administration system operates with the set operating parameters.

The control circuitry can be configured to set the operating parameter of the drug administration system by changing the operating parameter of the drug administration system to the first value from a different value. However, in some examples, it might not be necessary to change the operating parameter. For example, the control circuitry can check a value of the operating parameter of the drug administration system, and if the value of the operating parameter matches the configuration data, then the control circuitry can set the value of the operating parameter by confirming the value without changing the value of the operating parameter.

In some examples, such as in a multi-dose dosing regimen or a multi-drug dosing regimen, the drug administration system can comprise a second reservoir configured to contain a second drug therein, where the second reservoir including a second data storage component. The second drug can be the same drug as the first drug or can be a different drug. The reader can be configured to read, from second data storage component, second configuration data for configuring the operating parameter of the drug administration system. Further, the control circuitry can be configured to set the operating parameter of the drug administration system to a second value based on the configuration data so that, when the pump assembly drives the drug from the second reservoir, the drug administration system operates with the operating parameter set to the second value.

The configuration data can relate to volume, dose amount, or delivery or flow rate as discussed above or to any other suitable operating parameter. For instance, the configuration data can relate to an identification of the drug within the reservoir. The control circuitry 36 can use the identification information to verify whether the drug in the reservoir is a counterfeited or unauthorized drug. For example, prior to delivery initiation, the pump could engage the data storage component in a “challenge-response” authentication process to identify use of counterfeited or unauthorized drugs. The control circuitry 36 can be configured to set an operating parameter of the drug administration system based on the detection of a counterfeited or unauthorized drug to (i) shut off the drug administration system so that the drug administration system cannot deliver the drug and/or (ii) provide an alarm or warning to the operator about the counterfeited or unauthorized drug.

The control circuitry 36 can use the identification information in a multi-sequence delivery to verify whether the drug loaded into the pump is loaded in the proper sequence. In such examples, the control circuitry 36 can be configured to set an operating parameter of the drug administration system based on the detection of an improper drug sequence to (i) shut off the drug administration system so that the drug administration system cannot deliver the drug and/or (ii) provide an alarm or warning to the operator about the improper drug sequencing.

The configuration data can relate to the expiration date of the drug in the reservoir. In such examples, the control circuitry 36 can be configured to set an operating parameter of the drug administration system based on the detection of an expired drug to (i) shut off the drug administration system so that the drug administration system cannot deliver the expired drug and/or (ii) provide an alarm or warning to the operator about the expired drug.

The configuration data can relate to a desired delivery force to be used to deliver the drug stored in the reservoir, and an operating parameter of the pump (e.g., parameter of the motor) can be set to deliver the drug using the desired delivery force. For example, a high-viscosity drug may be more difficult to deliver at a specific flow rate than a low-viscosity drug, and the high-viscosity drug may need to be dispensed from the pump using higher forces (e.g., exerting higher forces on the pump's plunger 34), higher pressures within the pumping chamber 28, and/or more power from the pump's power supply 420. The control circuitry 36 that monitors the pump's operation and detects failures may therefore be programmed in response to configuration data read from the data storage component with a different set of thresholds to assess proper pump operation during delivery of a high viscosity drug. These thresholds can be preprogrammed in the data storage component, so that they can be implemented in the pump's hardware and software prior to the start of the drug delivery. Alternatively, these thresholds can be preprogrammed in the control circuitry 36, and the control circuitry 36 can select the appropriate thresholds based on the configuration data stored on the data storage component 48 (e.g., identification of the drug).

As a specific example, a desired plunger force range during delivery of a low-viscosity drug might be 10 to 15 N, and a force outside this range would be considered abnormal operation and result in an alarm. By comparison, a desired plunger force range of a higher-viscosity drug may be 20 to 30 N. Each data storage component can be preprogrammed with data relating to the desired operating parameters for the associated drug, and the control circuitry 36 can reconfigure the pump for the desired operating ranges. The control circuitry 36 can reconfigure the pump by changing software thresholds and/or selecting different software algorithms. The control circuitry 36 can also reconfigure hardware by modifying the settings of various hardware components, such as (without limitation) digital potentiometers, electronic range-switching circuits, sensors, transducers, and/or amplifiers.

The configuration data can relate to a desired temperature of the drug at time of delivery. For example, a particular drug may require refrigerated storage and require that the drug be warmed to a specified temperature before the pump begins delivery. The temperature information can be stored on the data storage component, and the pump can be equipped with a temperature-measuring component, such as a thermistor. The control circuitry 36 can be configured to detect a temperature of the drug, and to set an operating parameter of the drug administration system based on the detection of the temperature of the drug. In some examples, the control circuitry 36 can (i) shut off the drug administration system until the drug has warmed to a desired temperature and/or (ii) provide an alarm or warning to the operator that the drug has not warmed to a desired temperature.

The configuration data can store information that the control circuitry 36 uses to modify the pump's user interface behavior in accordance with the associated drug. For example, upon reading a data storage component, the pump's display could inform the user of special instructions/warnings associated with a particular drug, and/or instructions on how to prepare the next drug in a manually-implemented multi-vial sequence. Similarly, the control circuitry 36 can adjust the operation of other user interface devices such as (without limitation) tactile buzzers, speakers, piezo transducers, and/or LEDs to create special alerts and warnings associated with the delivery of a particular drug or delivery of a sequence of drugs.

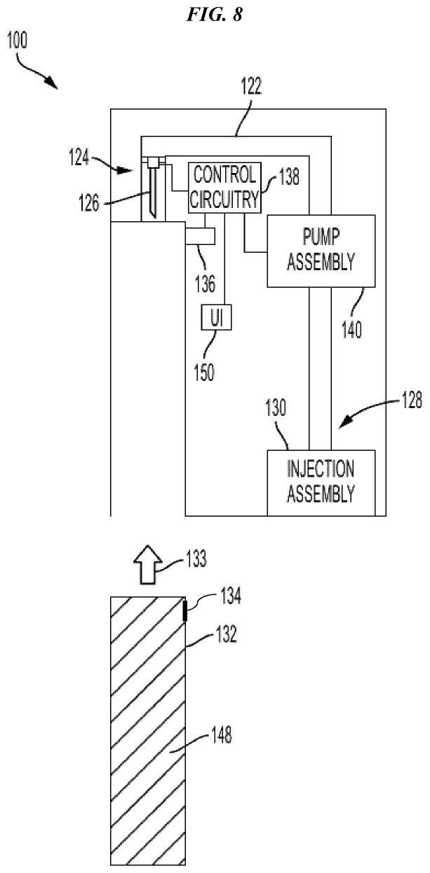

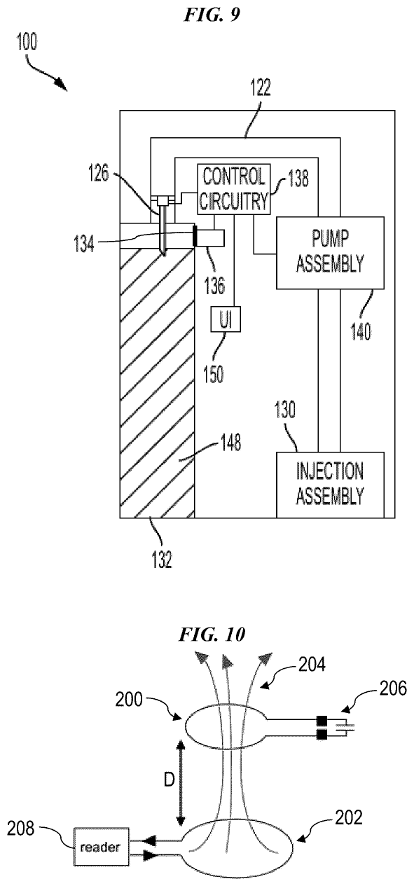

FIGS. 8 and 9 illustrate another embodiment of a pump 100 configured to deliver a liquid drug 148 to the patient. The pump 100 of FIGS. 8 and 9 is generally configured and used similar to the pump 20 of FIG. 1. The pump 100 is configured to engage with a therapeutic substance reservoir 132. The reservoir 132 can have any of a variety of configurations, similar to that discussed above regarding the reservoir 24 of FIG. 1. Within the pump 100 is a sterile fluid path 122 for delivering the drug 148 to a patient wearing the pump 100. The sterile fluid path 122 has a conduit 126 at an upstream end 124 of the sterile fluid path 122 and has an injection assembly (also referred to herein as an “injector assembly”) 130 at a downstream end 128 of the sterile fluid path 122.

The pump 100 also includes control circuitry 138, a user interface (UI) 150 operably coupled to the control circuitry 138 and configured to provide information to a user of the pump 100, and a reader 136 operably coupled to the control circuitry 138 and configured to read a data storage component 134 of the reservoir 132. The user interface 150 can have any of a variety of configurations, similar to that discussed above regarding the user interface 40 of FIG. 1. The data storage component 148 and the reader 136 can each have a variety of configurations, similar to that discussed above regarding the data storage component 48 and the reader 50 of FIG. 1. The control circuitry 136 is configured to control administration of the drug 148 from the pump 100 according to a dosing regimen similar to that discussed above regarding the control circuitry 36 of FIG. 1. The control circuitry 136, e.g., a processor thereof, is thus configured to establish at least one of the algorithm's one or more variable parameters based on the data received from the reader 136 so as to be based at least in part on the data stored on the data storage component 134 of the reservoir 132.

The pump 100 and the reservoir 132 are configured to engage with one another, such as shown by the reservoir 132 being moved into a housing 110 of the pump 100 in a direction of an arrow 133 shown in FIG. 8. The reservoir 132 is thus configured to be inserted into the pump 100. Similar to that discussed above regarding the pump 20 of FIG. 1, the reservoir 132 can be non-removably received in the pump 100 or can be removably and replaceably received in the pump 100. The conduit 126 is configured to be driven to penetrate the reservoir 132 when the pump 100 and the reservoir 132 are engaged with one another, such that fluid communication is established between the reservoir 132 and the sterile fluid path 122, as is shown in FIG. 9. In embodiments in which the data storage component 134 is located on a top of the reservoir 132 through which the conduit 126 pierces, the conduit 126 can be configured to pierce through the data storage component.

Once fluid communication is established between the reservoir 132 and the sterile fluid path 122, and the control circuitry 138 has adjusted the dosing regimen based on the data received from the reader 136, the control circuitry 138 is configured to drive a pump assembly 140 of the pump 100 to draw the drug 148 from the reservoir 132 and deliver the drug 148 to the patient via the injection assembly 130 similar to that discussed above regarding the control circuitry 36 and the injection assembly 46 of FIG. 1.

The data storage component 134 is located on a side of the reservoir 132 in this illustrated embodiment but, as discussed above, the reservoir 132 can include the data storage component 134 at another location. The reader 136 is located within the pump 100 so as to be configured to align with the data storage component 134 with the reservoir 132 received in the pump 100, as shown in FIG. 9.

In general, in embodiments in which a data storage component includes an NFC tag, the data storage component of the reservoir received in the pump is located within an effective distance of the pump's reader. In this way, the reader can effectively read data from the NFC tag. In some embodiments, regardless of the reservoir's alignment relative to the pump, the reader is within an effective distance of the pump. For example, if the data storage component is located on a top of a reservoir or on a bottom of a reservoir (e.g., as shown in FIG. 3 in which the NFC tag 72 is located on a bottom of the vial 70), a rotational alignment of the reservoir relative to the pump will not affect the reader's effective distance from the data storage component. In other embodiment, alignment of the reservoir relative to the pump can affect the reader's ability to effectively read the data storage component. In such embodiments, minimizing the distance between the pump's reader and the reservoir's data storage component may help ensure that the reader is within the effective distance to properly read data from the data storage component.

In an exemplary embodiment, the pump's reader and the reservoir's data storage component are at a minimal distance from one another and having windings substantially coaxially aligned and normal in a same plane and of substantially the same shape and size and with a maximum number of electromagnetic field lines generated by the reader, e.g., by an antenna thereof, passing through the data storage component, e.g., through an antenna thereof. A person skilled in the art will appreciate that axes may not be precisely coaxially aligned, that planes may not be precisely normal, and that sizes and shapes may not be precisely the same but nevertheless be considered to be substantially coaxially aligned, substantially normal, or substantially of the same size and shape due to any number of factors, such as manufacturing tolerances and sensitivity of measurement equipment.

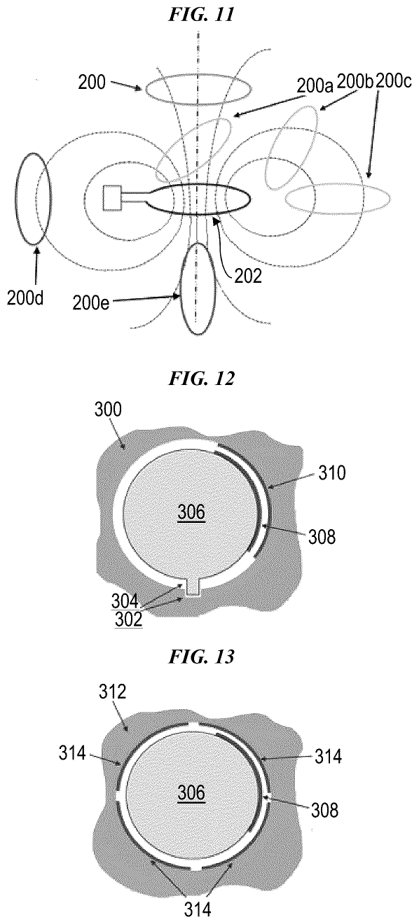

FIG. 10 illustrates one embodiment of relative positioning of an NFC tag antenna 200 of a data storage component being substantially coaxially aligned and normal in a same plane and of substantially the same shape and size as an NFC reader antenna 202 of a reader. A maximum number of electromagnetic field lines 204 generated by the NFC reader antenna 202 pass through the NFC tag antenna 200, which is at a minimal distance D from the NFC reader antenna 202. The data storage component in this illustrated embodiment also includes an NFC tag chip 206 operatively coupled to the NFC tag antenna 200, and the reader in this illustrated embodiment also includes a reader chip 208 operatively coupled to the NFC reader antenna 202. FIG. 11 shows a comparison of the NFC tag antenna 200 and the NFC reader antenna 202 of FIG. 10 with less desirable positions and orientations of the NFC tag antenna 200a, 200b, 200c, 200d, 200e relative to the NFC reader antenna 202.

A pump and a reservoir configured to be received in the pump can include cooperating alignment mechanisms configured to facilitate optimal performance of the pump's reader and the reservoir's data storage component by cooperating to optimally position the reader and the data storage component relative to one another. The cooperating alignment mechanisms can have a variety of configurations.

FIG. 12 illustrates one embodiment in which a pump 300 includes a female alignment mechanism 302 configured to engage a male alignment mechanism 304 of a reservoir 306, which is a syringe in this illustrated embodiment but can be of another type as discussed herein. In other embodiments, the pump 300 can include a male alignment mechanism and the reservoir 306 can include a female alignment mechanism. This illustrated embodiment includes a single male alignment mechanism 302 and a single female alignment mechanism 304, but another, equal number of cooperating male and female alignment mechanisms can be used.

The alignment mechanisms 302, 304 are configured to slidably engage when the reservoir 306 is being inserted into the pump 300. Without the alignment mechanisms 302, 304 being engaged with one another, e.g., without the male alignment mechanism 304 being seated in the female alignment mechanism 302, the reservoir 306 cannot be inserted into the pump 300 because the space within the pump 300 for the reservoir 306 is too small for the reservoir 306 to be inserted into the pump 300. The reservoir 306 can therefore only be inserted into the pump 300 in a predetermined orientation relative to the pump 300. A data storage component 308, e.g., an antenna thereof, of the reservoir 306 will thus be positioned at an effective location relative to a reader 310, e.g., an antenna thereof, of the pump 300 with the reservoir 306 received in the pump 300, as shown in FIG. 12. The data storage component 308 is on a cylindrical side of the reservoir 306 in this illustrated embodiment, but cooperating alignment mechanisms can be similarly used with a reservoir's data storage component at another location.

As mentioned above, in some embodiments, a pump's reader can include a multiplexed reader, in which case a reservoir's data storage component has multiple effective positions relative to the reader. In such embodiments, cooperating alignment mechanisms need not be used. Using the embodiment of the reservoir 306 of FIG. 12 by way of example, FIG. 13 illustrates one embodiment of a pump 312 including a multiplexed reader including a plurality of reader antennas 314. FIG. 13 shows the reservoir 306 inserted into the pump 312.

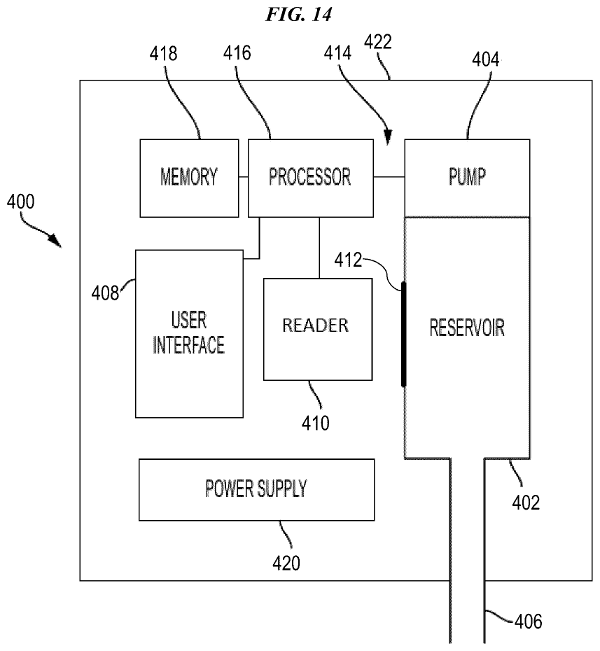

FIG. 14 illustrates another embodiment of a pump 400 configured to deliver a drug to the patient. The pump 400 of FIG. 14 is generally configured and used similar to the pump 20 of FIG. 1. The pump 400 includes a reservoir 402 configured to contain a liquid drug therein to be delivered from the pump 400. The reservoir 402 can have a variety of configurations, as discussed herein. The pump 400 also includes a pumping assembly 404 configured to cause dispensing of the drug contained in the reservoir 402 so that the drug can be delivered to the patient. The pump 400 also includes an injector assembly that includes an infusion line 406, e.g., a needle or a cannula, configured to be removably attached to a patient. The drug is delivered from the reservoir 402 upon actuation of the pumping assembly 404 via the infusion line 406. The pump 400 in the illustrated embodiment of FIG. 14 is thus configured as an “off body” pump. In some embodiments, the pump 400 is a single-use pump with the pump 400 only being used until the infusion line 406 is removed from the patient. In other embodiment, the pump 400 is a multi-use pump in which the pump 400 is reusable with different infusion lines 406.

The pump 400 also includes a user interface 408 configured to provide information to a user of the pump 400, and a reader 410 configured to read data from a data storage component 412 of the reservoir 402. The user interface 408, the reader 410, and the data storage component 412 can each have a variety of configurations, as discussed herein. The pump 400 and the reservoir 402 can include cooperating alignment mechanisms, as discussed herein.

The pump 400 also includes control circuitry 414 that includes a processor 416 and a memory 418. The processor 416 is operatively coupled to the memory 418, the user interface 408, the reader 410, and the pumping assembly 404. Actuation of the pumping assembly 404 is controlled by the processor 416.

The pump 400 also includes a power supply 420 configured to provide power to any components of the pump 400 that require power for operation, such as the pumping assembly 404, the processor 416, and the user interface 408.

The reservoir 402, the pumping assembly 404, the user interface 408, the power supply 420, and the control circuitry 414 are located within a housing (also referred to herein as a “body” of a pump) 422 of the pump 400. The infusion line 406 is partially located within the housing 422 and extends from the housing 422 for penetration into the patient. The infusion line 406 can be fixedly positioned partially within the housing 422 and partially outside the housing 422, as shown in FIG. 14, or the infusion line 406 can be movable, e.g., under control of the circuitry 414, from an initial position entirely within the housing 422 to a delivery position partially within the housing 422 and partially outside the housing 422.

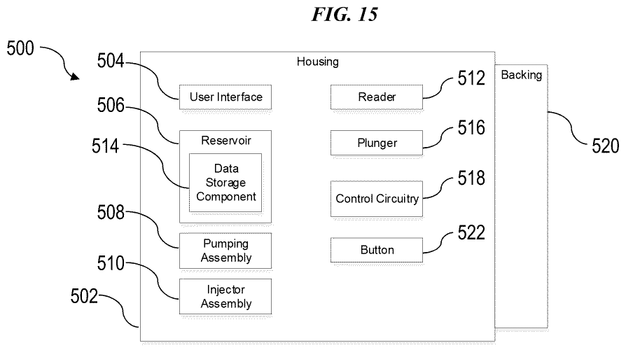

FIG. 15 illustrates another embodiment of a pump 500 configured to be worn by a patient and to deliver a liquid drug to the patient. The pump 500 of FIG. 15 is generally configured and used similar to the pump 20 of FIG. 1, e.g., includes a housing 502, a user interface 504, a reservoir 506 configured to contain a liquid drug therein to be delivered from the pump 500, a pumping assembly 508 configured to cause dispensing of the drug contained in the reservoir 506, an injector assembly 510 configured to deliver the drug into the patient, a reader 512 configured to read a data storage component 514 of the reservoir 506, a plunger 516 configured to slide within the pump chamber, and control circuitry 518 operatively connected to the reader 512, the user interface 504, and the pumping assembly 508.

The pump 500 also includes a removable backing or label 520 and a depressible button 522. The backing or label 520 is configured to be removed from the housing 502 of the pump 500 to expose adhesive attachable to the patient to allow the pump 500 to be removably attached to the patient. The button 522 is configured to be pressed by a user to start operation of the pump 500 to deliver the drug to the patient according to the stored dosing regimen.

The user interface 504 in this illustrated embodiment includes a plurality of lights that are arranged circumferentially around the button 522. The lights can be illuminated to provide various information to the user. The lights includes a plurality of lights, e.g., two, three, four, five, etc., in this illustrated embodiment but can include a single light. Instead of or in addition to surrounding the button 522, the light(s) can be, for example, arranged in a line. Various embodiments of lights and light illuminations to provide information are described further in, for example, previously mentioned Intl. Pat. Pub. WO 2021/124002 entitled “Liquid Drug Pumps Including User Feedback Indicating Pump Orientation” published Jun. 24, 2021.

The present disclosure has been described above by way of example only within the context of the overall disclosure provided herein. It will be appreciated that modifications within the spirit and scope of the claims may be made without departing from the overall scope of the present disclosure. All publications and references cited herein are expressly incorporated herein by reference in their entirety for all purposes.

What is claimed is:

1. A drug administration system, comprising:
a first reservoir containing a first volume of a first drug therein and including a first data storage component storing data indicative of the first volume;
a second reservoir having a same size as the first reservoir, containing a second volume of a second drug therein, and including a second data storage component storing data indicative of the second volume, the second volume being different from the first volume; and
a pump configured to receive each of the first and second reservoirs therein, the pump comprising:
a reader configured to read the stored data from the first and second data storage components;
a pumping assembly configured to drive the first drug from the pump for delivery to a patient and to drive the second drug from the pump for delivery to the patient, and
control circuitry operably coupled to the reader and to the pumping assembly and configured to
receive first data from the reader indicative of the stored data read from the first data storage component,
establish a first dosing regimen for delivery of the first drug using the received first data,
cause the pumping assembly to drive the first drug from the pump based on the first dosing regimen,
receive second data from the reader indicative of the stored data read from the second data storage component,
establish a second dosing regimen for delivery of the second drug using the received second data, and
cause the pumping assembly to drive the second drug from the pump based on the second dosing regimen.

2. The system of claim 1, wherein the control circuitry is configured to establish the first dosing regimen prior to any delivery of the first drug from the pump to the patient; and
the control circuitry is configured to establish the second dosing regimen prior to any delivery of the second drug from the pump to the patient.

3. The system of claim 1, wherein:
the reader includes a Near Field Communication (NFC) reader, the first data storage component includes a first NFC tag, and the second data storage component includes a second NFC tag; or
the reader includes a QR code scanner, the first data storage component includes a first QR code, and the second data storage component includes a second QR code.

4. (canceled)

5. The system of claim 1, wherein the first data storage component includes a first electrically erasable programmable read-only memory (EEPROM), and the second data storage component includes a second EEPROM.

6. The system of claim 1, wherein the first drug is the same as the second drug.

7. The system of claim 1, wherein the first drug is different from the second drug.

8. The system of claim 1, wherein the first and second reservoirs are configured to be pre-loaded into the pump.

9. The system of claim 1, wherein the first and second reservoirs are configured to be user-loaded into the pump.

10. The system of claim 1, wherein the first reservoir is configured to be removed from the pump before the second reservoir is received in the pump.

11. A drug administration method, comprising:
a reader of a pump reading stored data from first and second data storage components;
control circuitry of the pump establishing a first dosing regimen and causing a pumping assembly of the pump to drive a first drug from a first reservoir in the pump based on the first dosing regimen; and
the control circuitry establishing a second dosing regimen and causing the pumping assembly to drive a second drug from a second reservoir in the pump based on the second dosing regimen;
wherein the first reservoir contains a first volume of the first drug therein and includes the first data storage component storing data indicative of the first volume;
the second reservoir has a same size as the first reservoir, contains a second volume of the second drug therein, and includes the second data storage component storing data indicative of the second volume;
the second volume is different from the first volume; and
the control circuitry receives first data from the reader indicative of the stored data read from the first data storage component, establishes the first dosing regimen using the received first data, receives second data from the reader indicative of the stored data read from the second data storage component, and establishes the second dosing regimen using the received second data.

12. The method of claim 11, wherein the control circuitry establishes the first dosing regimen prior to any delivery of the first drug from the pump to the patient, and the control circuitry establishes the second dosing regimen prior to any delivery of the second drug from the pump to the patient.

13. The method of claim 11, wherein;
the reader includes a Near Field Communication (NFC) reader, the first data storage component includes a first NFC tag, and the second data storage component includes a second NFC tag; or
the reader includes a QR code scanner, the first data storage component includes a first QR code, and the second data storage component includes a second QR code.

14. (canceled)

15. The method of claim 11, wherein the first data storage component includes a first electrically erasable programmable read-only memory (EEPROM), and the second data storage component includes a second EEPROM.

16. The method of claim 11, wherein the first drug is the same as the second drug.

17. The method of claim 11, wherein the first drug is different from the second drug.

18. The method of claim 11, wherein each of the first and second reservoirs is pre-loaded into the pump.

19. The method of claim 11, wherein each of the first and second reservoirs is user-loaded into the pump.

20. The method of claim 11, wherein the first volume of the first drug is delivered to the patient in the first dosing regimen, and the second volume of the second drug is delivered to the patient in the second dosing regimen.

21. (canceled)

22. (canceled)

23. A drug administration system, comprising:
a pump including a housing, a reader, and control circuitry; and
a reservoir configured to contain a drug therein, the reservoir including a data storage component configured to be preprogrammed, prior to the reservoir being received in the housing of the pump, with data indicating a dose amount of the drug to be delivered to a patient using the pump;
wherein the reader is configured to, with the reservoir received in the housing of the pump, read the data indicating the dose amount from the data storage component;
the control circuitry is configured to receive data from the reader indicative of the read data indicating the dose amount; and
the control circuitry is configured to cause a dose of the drug, at the dose amount, to be delivered from the pump to the patient.

24. The system of claim 23, wherein the dose amount is based on at least one of a pre-identified therapy and a pre-identified prescription of the drug for the patient.

25. The system of claim 23, wherein the dose amount is based on a pre-identified weight of the patient.

26. The system of claim 23, wherein the dose amount is based on at least one of gender, ethnicity, and genetic makeup.

27-35. (canceled)

36. A drug administration method, comprising:
a reader of a pump, with a reservoir received in a housing of the pump, reading data indicating a dose amount from a data storage component of the reservoir; and
control circuitry of the pump causing a dose of the drug, at the dose amount, to be delivered from the pump to the patient;
wherein the reservoir contains the drug therein;
the data storage component is preprogrammed, prior to the reservoir being received in the housing of the pump, with data indicating the dose amount of the drug to be delivered to the patient using the pump; and
the control circuitry receives data from the reader indicative of the read data indicating the dose amount.

37-80. (canceled)
